(19) United States
(12) Patent Application Publication
(10) Pub. No.: US 2009/0291919 A1
(43) Pub. Date: Nov. 26, 2009

(54) Compositions and Methods for Treating or Preventing Ophthalmic Light Toxicity

(75) Inventors: Shalesh Kaushal, Gainesville, FL (US); Syed M. Noorwez, Gainesville, FL (US)

Correspondence Address: Jeffrey D. Hsi;Edwards Angell Palmer & Dodge LLP, P.O. Box 55874, Boston, MA, 02205, US

(73) Assignee: University of Florida Research Foundation, Inc., Gainesville, FL (US)

(21) Appl. No.: 12/357,036

(22) Filed: Jan. 21, 2009

Related U.S. Application Data
(63) Continuation of application No. PCT/US2007/016992, filed on Jul. 27, 2007.
(60) Provisional application No. 60/833,884, filed on Jul. 27, 2006.
(60) Provisional application No. 60/878,492, filed on Jan. 3, 2007.
(60) Provisional application No. 60/933,430, filed on Jun. 5, 2007.

(51) Int. Cl. A61K 31/07; A61K 31/355; A61K 31/375; A61K 31/415; A61K 31/44; A61K 31/506; A61K 31/4965; A61K 31/662; A61K 31/13; A61K 31/12

(52) U.S. Cl. 514/75; 514/725; 514/458; 514/474; 514/406; 514/352; 514/274; 514/255.02; 514/663; 514/690

(57) ABSTRACT

Methods are disclosed for treating ophthalmic conditions related to the production of toxic visual cycle products that accumulate in the eye, and are associated with reactions of the visual cycle during medical procedures that expose the eye to light, most commonly the various forms of ophthalmic surgery. Compounds and compositions useful in the these methods, either alone or in combination with other therapeutic agents, are also described, along with methods of screening for new agents useful in said treatments.

### CROSS-REFERENCE TO RELATED APPLICATION

This application is related to U.S. Provisional Patent Applications 60/833,884, filed 27 Jul. 2006, 60/878,492, filed 3 Jan. 2007, and 60/933,430, filed Jun. 5, 2007, the disclosures of which are hereby incorporated by reference in their entirety.

### FIELD OF THE INVENTION

The present invention relates to methods of using opsin-binding agents for the treatment and/or prevention of light toxicity to the eye, such as that occurring during ophthalmic surgery and methods of screening for agents useful therefor.

### BACKGROUND OF THE INVENTION

The visual cycle (also frequently referred to as the retinoid cycle) comprises a series of light-driven and/or enzyme catalyzed reactions whereby a light-sensitive chromophore (called rhodopsin) is formed by covalent bonding between the protein opsin and the retinoid agent 11-cis-retinal and subsequently, upon exposure to light, the 11-cis-retinal is converted to all-trans-retinal, which can then be regenerated into 11-cis-retinal to again interact with opsin. A number of visual, ophthalmic, problems can arise due to interference with this cycle. It is now understood that at least some of these problems are due to improper protein folding, such as that of the protein opsin.

The main light and dark receptor in the mammalian eye is the rod cell, which contains a folded membrane containing protein molecules that can be sensitive to light, the main one being rhodopsin. Like other proteins present in mammalian cells, opsin is synthesized in the endoplasmic reticulum (i.e., on ribosomes) of the cytoplasm and then conducted to the cell membrane of rod cells.

The visual cycle comprises a series of enzyme catalyzed reactions, usually initiated by a light impulse, whereby the visual chromophore of rhodopsin, consisting of opsin protein bound covalently to 11-cis-retinal, is converted to an all-trans-isomer that is subsequently released from the activated rhodopsin to form opsin and the all-trans-retinal product. This part of the visual cycle occurs in the outer portion of the rod cells of the retina of the eye. Subsequent parts of the cycle occur in the retinal pigmented epithelium (RPE). Components of this cycle include various enzymes, such as dehydrogenases and isomerases, as well as transport proteins for conveying materials between the RPE and the rod cells.

As a result of the visual cycle, various products are produced, called visual cycle products. One of these is all-trans-retinal produced in the rod cells as a direct result of light impulses contacting the 11-cis-retinal moiety of rhodopsin. All-trans-retinal, after release from the activated rhodopsin, can be regenerated back into 11-cis-retinal or can react with an additional molecule of all-trans-retinal and a molecule of phosphatidyl ethanolamine to produce N-retinylidene-N-retinylethanolamine (dubbed “A2E”), an orange-emitting fluorophore that can subsequently collect in the rod cells and in the RPE. As A2E builds up (as a normal consequence of the visual cycle) it can also be converted into lipofuscin, a toxic substance that has been implicated in several abnormalities, including ophthalmic conditions such as macular degeneration. A2E can also prove toxic to the RPE and has been associated with macular degeneration.

Because the build-up of toxic visual cycle products is a normal part of the physiological process, it is likely that all mammals, especially all humans, possess such an accumulation to some extent throughout life. However, during surgical procedures on the eye, especially on the retina, where strong light is required over an extended period, for example, near the end of cataract surgery and while implanting the new lens, these otherwise natural processes can cause toxicity because of the build-up of natural products of the visual cycle. Because of this, there is a need for agents that can be administered prior to, during or after (or any combination of these) the surgical process and that has the effect of reducing the production of visual cycle products that would otherwise accumulate and result in toxicity to the eye, especially to the retina.

The present invention answers this need by providing agents and methods of use for treating and/or amelioration such conditions, if not preventing them completely. Importantly, such agents are not retinoids and thus are not tightly controlled for entrance into the rod cells, where visual cycle products otherwise accumulate. Therefore, such agents can essentially be titrated in as needed for prevention of toxic build-up of visual cycle products like all-trans-retinal. Such compounds compete with 11-cis-retinal to reduce all-trans-retinal by tying up the retinal binding pocket of opsin. Thus, the compounds provided by the present invention (or identified by the screening methods of the invention) have the advantage that there is no limit on the amount of 11-cis-retinal that is produced in the eye (thus not contributing to retinal degeneration). Instead, the formation of all-trans-retinal is limited and thereby the formation of A2E is also reduced.

Computer-assisted molecular docking has lead to the successful discovery of novel ligands for more than 30 targets (Shoichet et al. (2002)). This strategy has been applied primarily to enzymes, such as aldose reductase (Iwata et al. (2001), Bcl-2 (Enyedy et al. (2001), matriptase (Enyedy et al. (2001), adenovirus protease (Pang et al. (2001)), AmpC fl-lactamase, carbonic anhydrase (Gruneberg et al. (2002)), HPRTase (Freymann et al. (2000)), dihydrodipicolinate (Paiva et al. (2001)) and Cdk4 (Honma et al. (2001)). Improvements in docking algorithms and multiprocessor resources have improved the technique of molecular docking to a point where it can be applied to more challenging problems. For example, this approach has recently been applied to defining small molecules that target protein-protein interfaces, which are relatively broad and flat compared to easily targeted enzyme active sites.

### BRIEF SUMMARY OF THE INVENTION

The invention generally provides a method of reducing light toxicity in a mammalian eye, involving administering to the mammal an opsin-binding agent that is a retinoid that binds non-covalently to the opsin protein; or is a non-retinoid that binds reversibly to the opsin protein; thereby reducing light toxicity in the mammalian eye. In one embodiment, the retinoid or non-retinoid opsin-binding agent selectively binds (e.g., reversibly, covalently, non-covalently) to opsin. In another embodiment, the opsin-binding agent is a non-retinoid. In yet another embodiment, the opsin binding agent binds at or near the retinal binding pocket of the opsin protein. In yet another embodiment, the opsin-binding agent binds to the opsin protein so as to inhibit covalent binding of 11-cis-retinal to the opsin protein when the 11-cis-retinal is contacted with the opsin protein in the presence of the non-retinoid opsin-binding agent. In yet another embodiment, opsin-binding agent binds to the opsin in the retinal binding pocket of opsin protein or disrupts retinoid binding to the retinal binding pocket of opsin. In yet another embodiment, the opsin-binding agent binds to the opsin protein so as to inhibit covalent binding of 11-cis-retinal to the opsin protein. In yet another embodiment, the mammal is a human being. In yet another embodiment, light toxicity is associated with the level of a visual cycle product, such as a visual cycle product formed from 11-cis-retinal or all-trans-retinal, a toxic visual cycle product, visual cycle product is formed from all-trans-retinal, lipofuscin or N-retinylidene-N-retinylethanolamine (A2E). In another embodiment, the administering is topical administration, local administration (e.g., intraocular injection or periocular) or systemic administration (e.g., oral, injection). In yet another embodiment, the light toxicity is related to an ophthalmic procedure (e.g., ophthalmic surgery). In still another embodiment, the administering occurs prior to, during, or after the ophthalmic surgery. In still another embodiment, the method further involves administering to the mammal an effective amount of at least one additional agent selected from the group consisting of a proteasomal inhibitor, an autophagy inhibitor, a lysosomal inhibitor, an inhibitor of protein transport from the ER to the Golgi, an Hsp90 chaperone inhibitor, a heat shock response activator, a glycosidase inhibitor, and a histone deacetylase inhibitor. In yet another embodiment, the opsin-binding agent and the additional agent are administered simultaneously. In still another embodiment, the opsin-binding agent and the additional agent are each incorporated into a composition that provides for their long-term release. In another embodiment, the composition is part of a microsphere, nanosphere, or nano emulsion. In another embodiment, the further involves administering a mineral supplement, at least one anti-inflammatory agent, such as a steroid (e.g., any one or more of cortisone, hydrocortisone, prednisone, prednisolone, methylprednisolone, triamcinolone, betamethasone, beclamethasone and dexamethasone), or at least one anti-oxidant, such as vitamin A, vitamin C and vitamin E. In various embodiments, the opsin-binding agent, the anti-inflammatory agent, and/or the anti-oxidant are administered simultaneously. In still other embodiments, the opsin-binding agent is selected from the group consisting of 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β3-ionone, cis-1,3-dimethylcyclohexane, and a pharmaceutically acceptable salt, hydrate, or solvate thereof.

In another aspect, the invention provides a method of identifying a non-retinoid opsin-binding agent that reduces light toxicity in a mammalian eye, involving contacting a opsin-protein with a non-retinoid opsin-binding test compound in the presence of 11-cis-retinal and under conditions that promote the binding of the test compound and the 11-cis-retinal to the opsin protein; and determining a reversible reduction in rate of formation of rhodopsin relative to the rate when the test compound is not present, thereby identifying the test compound as a non-retinoid opsin-binding agent that reduces light toxicity in a mammalian eye. In one embodiment, the contacting occurs in a eukaryotic cell (e.g., mammalian cell, human cell) expressing the opsin protein. In another embodiment, the mammalian cell is in a mammalian eye at the time of the contacting. In yet another embodiment, the mammalian eye is exposed to a light source prior to, during, or following the contacting. In another embodiment, the test compound reversibly binds non-covalently to the retinal binding pocket of the opsin protein. In yet another embodiment, the test compound is selective for binding to opsin. In yet another embodiment, the non-retinoid opsin-binding agent binds to the opsin protein so as to inhibit covalent binding of 11-cis-retinal to the opsin protein when the 11-cis-retinal is contacted with the opsin protein when the non-retinoid opsin-binding agent is present.

### BRIEF DESCRIPTION OF THE DRAWINGS

FIG. 1A shows that when purified WT opsin was regenerated with 11-cis-retinal it formed a 500 nm absorbing pigment. FIG. 1B shows that formation of this pigment was inhibited by β-ionone, which (b) does not itself form a 500 nm absorbing pigment with opsin.

FIG. 2A shows that pigment formation of WT opsin with 11-cis-retinal was inhibited by SN10011 at 2 mM and 5 mM concentrations. FIG. 2B shows that no 500 nm absorbing pigment was generated by SN10011 with 11-cis-retinal in vitro. FIG. 2C shows that the SN10011 compound does not absorb in the visible spectrum.

FIG. 3 shows the molecular docking strategy for the compounds of the invention. FIG. 3A shows the retinal binding pocket of human opsin.

FIG. 3B shows binding of β-ionone in the pocket. FIG. 3C shows binding of compound SN10011 in the retinal pocket.

### DEFINITIONS

Unless expressly stated otherwise elsewhere herein, the following terms have the stated meaning with respect to the present invention.

By “proteasomal inhibitor” is meant a compound that reduces a proteasomal activity, such as the degradation of a ubiquinated protein.

By “autophagy inhibitor” is meant a compound that reduces the degradation of a cellular component by a cell in which the component is located.

By “lysosomal inhibitor” is meant a compound that reduces the intracellular digestion of macromolecules by a lysosome. In one embodiment, a lysosomal inhibitor decreases the proteolytic activity of a lysosome.

By “Inhibitor of ER-Golgi protein transport” is meant a compound that reduces the transport of a protein from the ER (endoplasmic reticulum) to the Golgi, or from the Golgi to the ER.

By “HSP90 chaperone inhibitor” is meant a compound that reduces the chaperone activity of HSP90. In one embodiment, the inhibitor alters protein binding to an HSP90 ATP/ADP pocket.

By “heat shock response activator” is meant a compound that increases the chaperone S activity or expression of a heat shock pathway component. Heat shock pathway components include, but are not limited to, HSP100, HSP90, HSP70, HASP60, HSP40 and small HSP family members.

By “glycosidase inhibitor” is meant a compound that reduces the activity of an enzyme that cleaves a glycosidic bond.

By “histone deacetylase inhibitor” is meant a compound that reduces the activity of an enzyme that deacetylates a histone.

By “reduces” or “increases” is meant a negative or positive alteration, respectively. In particular embodiments, the alteration is by at least about 10%, 25%, 50%, 75%, or 100% of the initial level.

As stated herein, the term “wild-type conformation” refers to the 3 dimensional conformation or shape of a protein that is free of mutations present in its amino acid sequence, such that protein function is altered relative to wild-type protein function. For opsin, a wild-type conformation is a conformation that is free from mutations that cause mis-folding, such as the mutation designated P23H (P23H opsin) (see, for example, GenBank Accession Nos. NM_{—}000539 and NP_{—}000530) (meaning that a proline is replaced by a histidine at residue 23 starting from the N-terminus). Opsin in a “wild-type conformation” is capable of opsin biological function, including but not limited to, retinoid binding, visual cycle function, and insertion into a photoreceptor membrane.

By “agent” is meant a small compound, polypeptide, polynucleotide, or fragment thereof.

By “correcting the conformation” of a protein is meant inducing the protein to assume a conformation having at least one biological activity associated with a wild-type protein.

By “mis-folded opsin protein” is meant a protein whose tertiary structure differs from the conformation of a wild-type protein, such that the misfolded protein lacks one or more biological activities associated with the wild-type protein.

By “opsin-binding agent” is meant a small molecule, polypeptide, or polynucleotide, or fragment thereof, capable of binding to an opsin polypeptide. In one embodiment, the agent is a retinoid that binds opsin non-covalently and reversibly. In another embodiment, the agent is a non-retinoid small compound that binds reversibly to opsin. The term “retinoid” refers to diterpenes having a non-aromatic 6-member ring core hydrocarbon structure and an eleven carbon side chain. Exemplary retinoids include 1-cis-retinal and all-trans-retinal.

“By “selectively binds” is meant a compound that recognizes and binds a polypeptide of the invention, but which does not substantially recognize and bind other molecules in a sample, for example, a biological sample.

By “effective amount” is meant a level of an agent sufficient to exert a physiological effect on a cell, tissue, or organ or a patient.

By “control” is meant a reference condition. In one embodiment, a cell contacted with an agent of the invention is compared to a corresponding cell not contacted with the agent.

By “treat” is meant decrease, suppress, attenuate, diminish, arrest, or stabilize the development or progression of a disease.

By “prevent” is meant reduce the risk that a subject will develop a condition, disease, or disorder.

By “competes for binding” is meant that a compound of the invention and an endogenous ligand are incapable of binding to a target at the same time. Assays to measure competitive binding are known in the art, and include, measuring a dose dependent inhibition in binding of a compound of the invention and an endogenous ligand by measuring t_{1/2}, for example.

As used herein, the term “pharmaceutically acceptable salt,’ is a salt formed from an acid and a basic group of one of the compounds of the invention (e.g., compounds in Example 1 or β-ionone or cis-1,3-dimethylcyclohexane)). Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbatc, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The term “pharmaceutically acceptable salt” also refers to a salt prepared from a compound of the invention (e.g., compounds in Example 1) having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl-N-ethylamine; diethylamine; triethylamine; mono-, bis-, or tris-(2-hydroxy-lower alkylamines), such as mono-, bis-, or tris-(2-hydroxyethyl)-amine, 2-hydroxy-tert-butylamine, or tris-(hydroxymethyl)methylamine, N,N, —di-lower alkyl-N-(hydroxy lower alkyl)-amines, such as N,N-dimethyl-N-(2-hydroxyethyl)-amine, or tri-(2-hydroxyethyl)amine; N-methyl-D-glucamine; and amino acids such as arginine, lysine, and the like.

The term “pharmaceutically acceptable salt” also refers to a salt prepared from a compound disclosed herein, e.g., a compound of Example 1, having a basic functional group, such as an amino functional group, and a pharmaceutically acceptable inorganic or organic acid. Suitable acids include, but are not limited to, hydrogen sulfate, citric acid, acetic acid, oxalic acid, hydrochloric acid, hydrogen bromide, hydrogen iodide, nitric acid,

phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid, and p-toluenesulfonic acid.

The term “pharmaceutically-acceptable excipient” as used herein means one or more compatible solid or liquid tiller, diluents or encapsulating substances that are suitable for administration into a human.

The term “carrier” denotes an organic or inorganic ingredient, natural or synthetic, with which the active ingredient is combined to facilitate administration.

The term “parenteral” includes subcutaneous, intrathecal, intravenous, intramuscular, intraperitoncal, or infusion.

The term “visual cycle product” refers to a chemical entity produced as a natural product of one or more reactions of the visual cycle (the reactive cycle whereby opsin protein binds 11-cis-retinal to form rhodopsin, which accepts a light impulse to convert 11-cis-retinal to all trans-retinal, which is then released from the molecule to regenerate opsin protein with subsequent binding of a new 11-cis-retinal to regenerate rhodopsin). Such visual cycle products include, but are not limited to, all-trans-retinal, lipofuscin and A2E.

The term “light toxicity” refers to any condition affecting vision that is associated with, related to, or caused by the production and/or accumulation of visual cycle products. Visual cycle products include, but are not limited to, all-trans-retinal, lipofuscin or A2E. In one particular embodiment, light toxicity is related to exposure of the eye to large amounts of light or to very high light intensity, occurring, for example, during a surgical procedure on the retina.

The term “opsin” refers to an opsin protein, preferably a mammalian opsin protein, most preferably a human opsin protein. In one embodiment, the opsin protein is in the wild-type (i.e., physiologically active) conformation. One method of assaying for physiological activity is assaying the ability of opsin to bind 11-cis-retinal and form active rhodopsin. A mutant opsin, such as the P23H mutant, that is ordinarily mis-folded has a reduced ability to bind 11-cis-retinal, and therefore forms little or no rhodopsin. Where the conformation of the mutant opsin has been corrected (for example, by binding to a pharmacological chaperone), the opsin is correctly inserted into the rod cell membrane so that its conformation is the same, or substantially the same, as that of a non-mutant opsin. This allows the mutant opsin to bind 11-cis-retinal to form active rhodopsin. Therefore, the methods of the invention operate to reduce the formation of visual cycle products.

### DETAILED DESCRIPTION OF THE INVENTION

In accordance with the present invention, it has been found that certain agents are capable of reversibly binding covalently or non-covalently to opsin protein and inhibiting the binding of retinoids, most notably 11-cis-retinal, to an opsin retinal binding pocket. Such interference with retinal binding reduces the formation of visual cycle products, such as all-trans-retinal, and thereby inhibits the production of compounds such as lipofuscin and A2E with resulting reduced risk and occurrence of toxicity that can result from accumulation of these substances.

Certain synthetic retinoids (compounds structurally related to retinol (Vitamin A alcohol)) have been reported to bind to opsin. In the embodiments of the present invention, opsin-binding agents are not synthetic or naturally-occurring retinoids (that is, the opsin-binding agents are not structurally analogous to retinol or retinal, e.g., the opsin-binding agents of the invention may lack a polyene chain and/or may lack a trimethylcyclohexene moiety. For purposes of this invention, beta-ionone is considered a non-retinoid and, in certain embodiments, is contemplated for use in the inventive methods and compositions. In certain embodiments, an opsin-binding agent is a non-polymeric (e.g., a small molecule) compound having a molecular weight less than about 1000 daltons, less than 800, less than 600, less than 500, less than 400, or less than about 300 daltons.

The invention features compositions and methods that are useful for reducing formation of visual cycle products and toxicity associated with the accumulation of such products in vivo.

The invention is generally based on the discovery that certain non-retinoid compounds can be used to prevent or reduce formation of visual cycle products and thereby reduce the incidence of toxicity caused by such accumulation. These compounds, which may also stabilize mutant opsin by binding to the opsin, e.g., at or near the retinal binding site (which includes binding in the retinal binding pocket), reversibly bind non-covalently and inhibit 11-cis-retinal from binding to the pocket, thereby reducing formation of products such as all-trans-retinal.

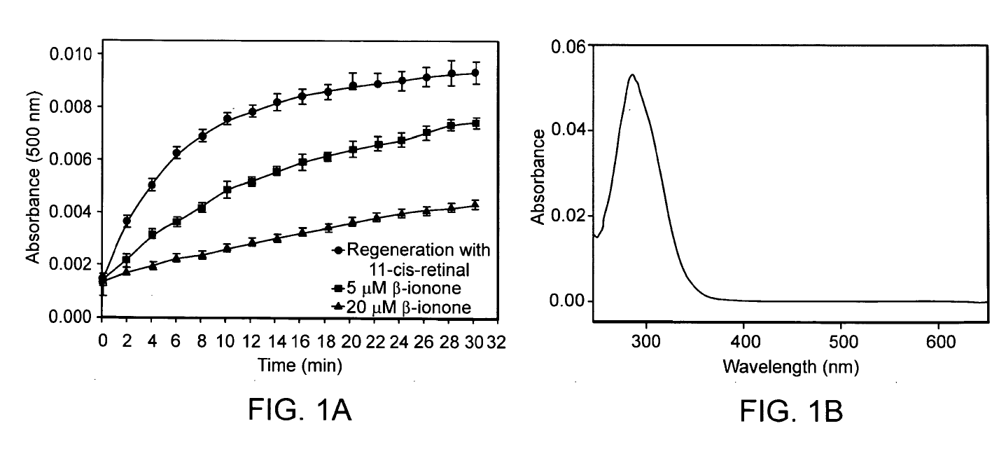

Opsin, the GPCR (G-protein coupled receptor) responsible for vision, readily regenerates with 11-cis-retinal to form the visual pigment rhodopsin. The pigment is generated by formation of a protonated Schiff base between the aldehyde group of 11-cis-retinal and the ε-amino group of L-lysine in opsin (Matsumoto and Yoshizawa, Nature Dec. 11, 1975; 258(5535):523-6). β-ionone (structure in Example 4) carries the six-membered ring configuration of retinal but has a shorter side chain (Daemen, 1978, Nature Dec. 21-28, 1978; 276(5690):847-8) and hence effectively competes with 11-cis-retinal for the chromophore binding site (Matsumoto &Yoshizawa supra; Daemen supra, Kefalov 1999, J. Gen. Physiol 1999 March; 113(3):491-503). In accordance with the invention, experimental conditions were found where β-ionone (and other small molecules) inhibited opsin regeneration in a dose dependent manner demonstrating the competitive nature of the interaction (FIG. 1a). The t_{1/2} of pigment formation was determined in the presence and the absence of β-ionone (see Example 2). In the absence of β-ionone, pigment formation occurred with a t_{1/2} of 5 minutes, while the presence of β-ionone increased the t_{1/2} to 10 (5 μM) and 16 minutes (20 μM), respectively. The increase in t_{1/2} was taken as evidence that β-ionone competed with 11-cis-retinal for the binding site. Further, we determined that no 500 nm absorbing pigment was formed upon addition of β-ionone to purified WT or native opsin protein (FIG. 1b).

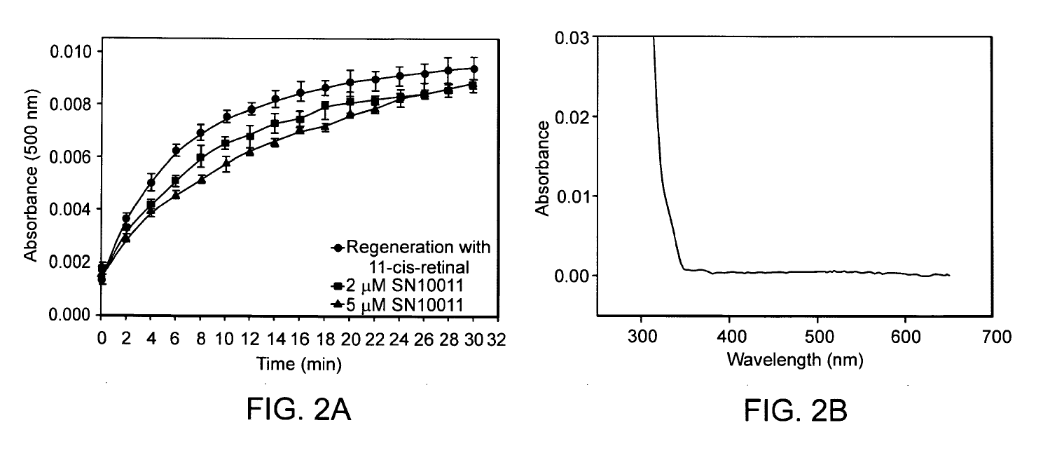

In accordance with the invention, similar results were found with other small organic molecules that were non-retinoids (see FIGS. 2a and 2b).

Thus, the present invention provides methods of discovery and use of small compounds that can fit into the retinal binding pocket of opsin and compete with 11-cis-retinal in vitro and thereby inhibit formation of 11-cis-retinal and other visual cycle products.

Molecular docking studies were used to identify candidate compounds that stabilize the retinal binding pocket of rhodopsin and that could be used for further study of the chemical and physical characteristics of such molecules for development of high throughput screening methods for compounds having therapeutic activity.

In accordance with the present invention, β-ionone interacts directly with the retinal binding pocket, so we docked β-ionone into the retinal binding pocket to determine the degree of structural complementarity necessary for enhancing rhodopsin folding. We utilized the crystal structure of rhodopsin to provide the basis for molecular docking and selected the site for molecular docking based on the position of retinal bound to rhodopsin. We then calculated a scoring grid base to encompass the region around the selected site for molecular docking, and subsequently used DOCK 5.1 (UCSF) to position β-ionone. The orientation of β-ionone posed by DOCK 5.1 showed that polar interactions and van der Waals contacts were involved in the specific interactions with rhodopsin.

To identify non-retinoid compounds that could be useful therapeutic agents, we performed molecular docking using a large chemical library of drug-like small molecules in the National Cancer Institute Developmental Therapeutics Program. DOCK5.1 (UCSF) was used to position each one of 20,000 drug-like compounds into the selected site. Each compound was positioned in 100 different orientations, and the best scoring orientations were obtained, Unlike previous molecular docking strategies, each docked compound was selected based on chemical criteria: the Lipinski rules for drug likeness. Therefore, this strategy eliminates compounds that are less likely to be developed into therapeutic agents. The fifth highest scoring compound was 1-(3,5-dimethyl-1H-pyrazol-4-yl)ethanone (Compound 1), SN10011, when in the orientation posed by DOCK5.1 (UCSF) at (including in) the retinal binding pocket based on the crystal structure of rhodopsin.

Methods of the Invention

The present invention provides a method of reducing the formation of toxic visual cycle products, comprising contacting an opsin protein with a non-retinoid opsin-binding agent that reversibly binds or a retinoid that binds non-covalently (for example, at or near the retinal binding pocket) to said opsin protein to inhibit retinoid binding in said binding pocket, thereby reducing formation of visual cycle products.

The present invention also provides a method of treating, preventing or reducing the risk of light toxicity in a mammal, comprising administering to a mammal, at risk of developing an ophthalmic condition that related to the formation or accumulation of a visual cycle product, an effective amount of an a non-retinoid opsin-binding agent that reversibly binds or a retinoid that binds non-covalently (for example, at or near the retinal binding pocket) to an opsin protein present in the eye of said mammal, for example, to inhibit retinoid binding in said binding pocket, thereby reducing light toxicity.

In specific examples of this method, the non-retinoid opsin-binding agent is selective for binding to opsin and/or the opsin-binding agent binds to said opsin in the retinal binding pocket of said opsin protein and/or the opsin-binding agent binds to said opsin protein so as to inhibit covalent binding of 11-cis-retinal to said opsin protein when said 11-cis-retinal is contacted with said opsin protein when said non-retinoid opsin-binding agent is present and/or the mammal is a human being.

In one embodiment, the light toxicity is related to an ophthalmic procedure, for example, ophthalmic surgery. Said agent may be administered prior to, during or after said surgery (or at any one or more of those times).

In specific embodiments of the methods of the invention, the native opsin protein is present in a cell, such as a rod cell, preferably, a mammalian and more preferably a human cell. In specific embodiments, the non-retinoid of the invention inhibits binding of 11-cis-retinal in the binding pocket of opsin and the visual cycle product whose formation is reduced or prevented is all-trans-retinal, or a toxic visual cycle product formed from it, such as lipofuscin or N-retinylidene-N-retinylethanolamine (A2E).

Non-limiting examples of compounds useful in the methods of the invention include 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, 1,3-dimethylcyclohexane, and a pharmaceutically acceptable salt thereof.

In methods of the invention, administering is preferably by topical administration (such as with an eye wash) or by systemic administration (including oral, intraocular injection or periocular injection). By way of preferred example, the ophthalmic condition to be treated is light toxicity, such as that resulting from ocular surgery, for example, retinal or cataract surgery.

Also encompassed is an opthalmologic composition comprising an effective amount of a non-retinoid opsin-binding agent in a pharmaceutically acceptable carrier, wherein said agent reversibly binds non-covalently (for example, at or near the retinal binding pocket) to said opsin protein to inhibit retinoid binding in said pocket, preferably where the non-retinoid opsin-binding agent is selective for opsin protein.

The present invention further provides a screening method for identifying a non-retinoid opsin-binding agent that reduces light toxicity in a mammalian eye, comprising:

(a) contacting a native opsin-protein with a non-retinoid opsin-binding test compound in the presence of 11-cis-retinal and under conditions that promote the binding of the test compound and the 11-cis-retinal to the native opsin protein; and

(b) determining a reversible reduction in rate of formation of rhodopsin relative to the rate when said test compound is not present,

thereby identifying said test compound as a non-retinoid opsin-binding agent that reduces light toxicity in a mammalian eye.

In a typical competition assay of the invention, a compound is sought that will tie up the retinal binding pocket of the opsin protein. Thus, the assay seeks to identify a non-retinoid compound (one that will not be tightly regulated by the retina as to amount entering rod cells) that competes with 11-cis-retinal. Over time this will slow the rate of formation of rhodopsin relative to the rate when 11-cis-retinal alone is present. In one embodiment, the assay is conducted in the presence of 11-cis-retinal, and the rate of formation of rhodopsin is measured as a way of determining competition for the retinal binding pocket, for example, by determining the rate of increase in the 500 nm peak characteristic for rhodopsin. No antibodies for rhodopsin are required for this assay. A useful compound will exhibit a rate of rhodopsin formation that is at least about 2 to 5 fold lower than that observed in the presence of 11-cis-retinal when said test compound is not present.

The opsin-binding agent may be administered along with other agents, including a mineral supplement, an anti-inflammatory agent, such as a steroid, for example, a corticosteroid, and/or an anti-oxidant. Among the corticosteroids useful for such administration are those selected from the group consisting of cortisone, hydrocortisone, prednisone, prednisolone, methylprednisolone, triamcinolone, betamethasone, beclamethasone and dexamethasone. Useful anti-oxidants include vitamin A, vitamin C and vitamin E.

The methods of the invention also contemplate reducing light toxicity by using at least one additional agent (in addition to the non-retinoid compound) selected from the group consisting of a proteasomal inhibitor, an autophagy inhibitor, a lysosomal inhibitor, an inhibitor of protein transport from the ER to the Golgi, an Hsp90 chaperone inhibitor, a heat shock response activator, a glycosidase inhibitor, and a histone deacetylase inhibitor, wherein the opsin-binding agent and the additional compound are administered simultaneously or within fourteen days of each other in amounts sufficient to treat the subject.

In a particular example of the methods of the invention, the opsin-binding agent and the additional compound are administered within ten days of each other, within five days of each other, within twenty-four hours of each other and preferably are administered simultaneously. In one example, the opsin-binding agent and the additional compound are administered directly to the eye. Such administration may be intra-ocular. In other examples, the opsin-binding agent and the additional compound are each incorporated into a composition that provides for their long-term release, such as where the composition is part of a microsphere, nanosphere, or nano emulsion.

As described herein, the opsin-binding agents useful in the methods of the invention and/or identified by any of the screening assays of the invention are available for use alone or in combination with one or more additional compounds to treat or prevent conditions associated with production and accumulation of visual cycle products, especially all-trans-retinal, such as light toxicity, for example, resulting from ocular surgical procedures. In one embodiment, a non-retinoid opsin-binding agent of the invention is administered without an additional-active compound. In another embodiment, a non-retinoid opsin-binding agent of the invention is used in combination with a synthetic retinoid (e.g., as disclosed in U.S. Patent Publication No. 2004-0242704), and optionally with another active compound (e.g., as discussed herein). In still another exemplary embodiment, an opsin-binding agent is administered in combination with the proteasomal inhibitor MG132, the autophagy inhibitor 3-methyladenine, a lysosomal inhibitor ammonium chloride, the ER-Golgi transport inhibitor brefeldin A, the Hsp90 chaperone inhibitor Geldamycin, the heat shock response activator Celastrol, the glycosidase inhibitor, and the histone deacetylase inhibitor Scriptaid, can be used to reduce formation of visual cycle products.

Proteasomal Inhibitors

The 26S proteasome is a multicatalytic protease that cleaves ubiquinated proteins into short peptides. MG-132 is one proteasomal inhibitor that may be used. MG-132 is particularly useful for the treatment of light toxicity and other ocular diseases related to the accumulation of visual cycle products (e.g., all-trans-retinal, A2E, lipofuscin), protein aggregation or protein misfolding. Other proteasomal inhibitors useful in the methods of the invention include lactocystin (LC), clasto-lactocystin-beta-lactone, PSI (N-carbobenzoyl-lle-Glu-(OtBu)-Ala-Leu-CHO), MG-132 (N-carbobenzoyl-Leu-Leu-Leu-CHO), MG-115 (Ncarbobenzoyl-Leu-Leu-Nva-CHO), MG-101 (N-Acetyl-Leu-Leu-norLeu-CHO), ALLM (NAcetyl-Leu-Leu-Met-CHO), N-carbobenzoyl-Gly-Pro-Phe-leu-CHO, N-carbobenzoyl-Gly-Pro-Ala-Phe-CHO, N-carbobenzoyl-Leu-Leu-Phe-CHO, and salts or analogs thereof. Other proteasomal inhibitors and their uses are described in U.S. Pat. No. 6,492,333.

Autophagy Inhibitors

Autophagy is an evolutionarily conserved mechanism for the degradation of cellular components in the cytoplasm, and serves as a cell survival mechanism in starving cells. During autophagy pieces of cytoplasm become encapsulated by cellular membranes, forming autophagic vacuoles that eventually fuse with lysosomes to have their contents degraded. Autophagy inhibitors may be used in combination with an opsin-binding or opsin-stabilizing compound. Autophagy inhibitors useful in the methods of the invention include, but are not limited to, 3-methyladenine, 3-methyl adenosine, adenosine, okadaic acid, N⁶-mercaptopurine riboside (N-MPR), an aminothiolated adenosine analog, 5-amino-4-imidazole carboxamide riboside (AICAR), bafilomycin A1, and salts or analogs thereof.

Lysosomal Inhibitors

The lysosome is a major site of cellular protein degradation. Degradation of proteins entering the cell by receptor-mediated endocytosis or by pinocytosis, and of plasma membrane proteins takes place in lysosomes. Lysosomal inhibitors, such as ammonium chloride, leupeptin, trans-epoxysaccinyl-L-leucylamide-(4-guanidino) butane, L-methionine methyl ester, ammonium chloride, methylamine, chloroquine, and salts or analogs thereof, are useful in combination with an opsin-binding or opsin-stabilizing compound.

HSP90 Chaperone Inhibitors

Heat shock protein 90 (Hsp90) is responsible for chaperoning proteins involved in cell signaling, proliferation and survival, and is essential for the conformational stability and function of a number of proteins. HSP-90 inhibitors are useful in combination with an opsin-binding or opsin-stabilizing compound in the methods of the invention. HSP-90 inhibitors include benzoquinone ansamycin antibiotics, such as geldanamycin and 17-allylamino-17-demethoxygeldanamycin (17-AAG), which specifically bind to Hsp90, alter its function, and promote the proteolytic degradation of substrate proteins. Other HSP-90 inhibitors include, but are not limited to, radicicol, novobiocin, and any Hsp90 inhibitor that binds to the Hsp90 ATP/ADP pocket.

Heat Shock Response Activators

Celastrol, a quinone metbide triterpene, activates the human heat shock response. In combination with an opsin-binding or opsin-stabilizing compound, celastrol and other heat shock response activators are useful for the treatment of PCD. Heat shock response activators include, but are not limited to, celastrol, celastrol methyl ester, dihydrocelastrol diacetate, celastrol butyl ester, dihydrocelastrol, and salts or analogs thereof.

Histone Deacetylase Inhibitors

Regulation of gene expression is mediated by several mechanisms, including the post-translational modifications of histones by dynamic acetylation and deacetylation. The enzymes responsible for reversible acetylationl/deacetylation processes are histone acetyltransferases (HATs) and histone deacetylases (HDACs), respectively. Histone deacetylase inhibitors include Scriptaid, APHA Compound 8, Apicidin, sodium butyrate, (−)-Depudecin, Sirtinol, trichostatin A, and salts or analogs thereof.

Glycosidase Inhibitors

Glycosidase inhibitors are one class of compounds that are useful in the methods of the invention, when administered in combination with an opsin-binding or opsin-stabilizing compound. Castanospermine, a polyhydroxy alkaloid isolated from plant sources, inhibits enzymatic glycoside hydrolysis. Castanospermine and its derivatives are particularly useful for the treatment of light toxicity or of an ocular Protein Conformation Disorder (PCD), such as retinitis pigmentosa. Also useful in the methods of the invention are other glycosidase inhibitors, including australine hydrochloride, 6-Acetamido-6-deoxy-castanosperrnine, which is a powerful inhibitor of hexosaminidases, Deoxyfuconojirimycin hydrochloride (DFJ7), Deoxynojirimycin (DNJ), which inhibits glucosidase I and II, Deoxygalactonojirimycin hydrochloride (DGJ), winch inhibits α-D-galactosidase, Deoxymannojirimycin hydrochloride (DM1), 2R,5R-Bis(hydroxymethyl)-3R,4R-dihydroxypyrrolidine (DMDP), also known as 2,5-dideoxy-2,5-imino-D-mannitol, 1,4-Dideoxy-1,4-imino-D-mannitol hydrochloride, (3R,4R,5R,6R)-3,4,5,6-Tetrahydroxyazepane Hydrochloride, which inhibits b-N-acetylglucosaminidase, 1,5-Dideoxy-1,5-imino-xylitol, which inhibits β-glucosidase, and Kifunensine, an inhibitor of mannosidase 1. Also useful in combination with an opsin-binding or opsin-stabilizing compound are N-butyldeoxynojirimycin (EDNJ), N-nonyl DNJ (NDND, N-hexyl DNJ (15TDNJ), N-methyldeoxynojirimycin (MDNJ), and other glycosidase inhibitors known in the art. Glycosidase inhibitors are available commercially, for example, from Industrial Research Limited (Wellington, New Zealand) and methods of using them are described, for example, in U.S. Pat. Nos. 4,894,388, 5,043,273, 5,103,008, 5,844,102, and 6,831,176; and in U.S. Patent Publication Nos. 20020006909.

Pharmaceutical Compositions

The present invention features pharmaceutical preparations comprising compounds together with pharmaceutically acceptable carriers, where the compounds provide for the inhibition of visual cycle products, such as all-trans-retinal or other products formed from 11-cis-retinal. Such preparations have both therapeutic and prophylactic applications. In one embodiment, a pharmaceutical composition includes an opsin-binding (e.g., a compound of Example 1, or β-ionone or 1,3-dimethylcyclohexane) or a pharmaceutically acceptable salt thereof; optionally in combination with at least one additional compound that is a proteasomal inhibitor, an autophagy inhibitor, a lysosomal inhibitor, an inhibitor of protein transport from the ER to the Golgi, an Hsp90 chaperone inhibitor, a heat shock response activator, a glycosidase inhibitor, or a histone deacetylase inhibitor. The opsin-binding or opsin-stabilizing compound is preferably not a natural or synthetic retinoid. The opsin-binding or opsin-stabilizing compound and the additional compound are formulated together or separately. Compounds of the invention may be administered as part of a pharmaceutical composition. The non-oral compositions should be sterile and contain a therapeutically effective amount of the opsin-binding or opsin-stabilizing compound in a unit of weight or volume suitable for administration to a subject. The compositions and combinations of the invention can be part of a pharmaceutical pack, where each of the compounds is present in individual dosage amounts.

The phrase “pharmaceutically acceptable” refers to those compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Non-oral pharmaceutical compositions of the invention to be used for prophylactic or therapeutic administration should be sterile. Sterility is readily accomplished by filtration through sterile filtration membranes (e.g., 0.2 μm membranes), by gamma irradiation, or any other suitable means known to those skilled in the art. Therapeutic opsin-binding or opsin-stabilizing compound compositions generally are placed into a container having a sterile access port, for example, an intravenous solution bag or vial having a stopper pierceable by a hypodermic injection needle. These compositions ordinarily will be stored in unit or multi-dose containers, for example, sealed ampoules or vials, as an aqueous solution or as a lyophilized formulation for reconstitution. The compounds may be combined, optionally, with a pharmaceutically acceptable excipient.

The components of the pharmaceutical compositions also are capable of being co-mingled with the molecules of the present invention, and with each other, in a manner such that there is no interaction that would substantially impair the desired pharmaceutical efficacy.

Compounds of the present invention can be contained in a pharmaceutically acceptable excipient. The excipient preferably contains minor amounts of additives such as substances that enhance isotonicity and chemical stability. Such materials are non-toxic to recipients at the dosages and concentrations employed, and include buffers such as phosphate, citrate, succinate, acetate, lactate, tartrate, and other organic acids or their salts; tris-hydroxymethylaminomethane (TRIS), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be included, such as stabilizers, anti-microbials, inert gases, fluid and nutrient replenishers (i.e., Ringer's dextrose), electrolyte replenishers, and the like, which can be present in conventional amounts.

The compositions, as described above, can be administered in effective amounts. The effective amount will depend upon the mode or administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

With respect to a subject suffering from, or at risk of developing, light toxicity, such as that due to ocular surgery, an effective amount is an amount sufficient to reduce the rate or extent of formation and accumulation of visual cycle products, such as all-trans-retinal, or lipofuscin, or A2E. Here, the compounds of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of a composition of the present invention.

A variety of administration routes are available. The methods of the invention, generally speaking, may be practiced using any mode of administration that is medically acceptable, meaning any mode that produces effective levels of the active compounds without causing clinically unacceptable adverse effects. In one preferred embodiment, a composition of the invention is administered intraocularly. Other modes of administration include oral, rectal, topical, intraocular, buccal, intravaginal, intracisternal, intracerebroventricular, intratracheal, nasal, transdermal, within/on implants, or parenteral routes. Compositions comprising a composition of the invention can be added to a physiological fluid, such as to the intravitreal humor. For CNS administration, a variety of techniques are available for promoting transfer of the therapeutic across the blood brain barrier including disruption by surgery or injection, drugs which transiently open adhesion contact between the CNS vasculature endothelial cells, and compounds that facilitate translocation through such cells. Oral administration can be preferred for prophylactic treatment because of the convenience to the patient as well as the dosing schedule.

Pharmaceutical compositions of the invention can optionally further contain one or more additional proteins as desired, including plasma proteins, proteases, and other biological material, so long as it does not cause adverse effects upon administration to a subject. Suitable proteins or biological material may be obtained from human or mammalian plasma by any of the purification methods known and available to those skilled in the art; from supernatants, extracts, or lysates of recombinant tissue culture, viruses, yeast, bacteria, or the like that contain a gene that expresses a human or mammalian plasma protein which has been introduced according to

standard recombinant DNA techniques; or from the fluids (e.g., blood, milk, lymph, urine or the like) or transgenic animals that contain a gene that expresses a human plasma protein which has been introduced according to standard transgenic techniques.

Pharmaceutical compositions of the invention can comprise one or more pH buffering compounds to maintain the pH of the formulation at a predetermined level that reflects physiological pH, such as in the range of about 5.0 to about 8.0 (e.g., 6.0, 6.5, 6.8, 6.9, 7.0, 7.1, 7.2, 7.3, 7.4, 7.5, 7.6, 7.8). The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g., tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) maybe present in any concentration sufficient to modulate the osmotic properties of the formulation.

Compositions comprising an opsin-binding or opsin-stabilizing compound of the present invention can contain multivalent metal ions, such as calcium ions, magnesium ions and/or manganese ions. Any multivalent metal ion that helps stabilize the composition and that will not adversely affect recipient individuals may be used. The skilled artisan, based on these two criteria, can determine suitable metal ions empirically and suitable sources of such metal ions are known, and include inorganic and organic salts.

Pharmaceutical compositions of the invention can also be a non-aqueous liquid formulation. Any suitable non-aqueous liquid may be employed, provided that it provides stability to the active agents (a) contained therein. Preferably, the non-aqueous liquid is a hydrophilic liquid. Illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425, PPG 725, PPG 1000, PEG 2000, PEG 3000 and PEG 4000.

Pharmaceutical compositions of the invention can also be a mixed aqueous/non-aqueous liquid formulation. Any suitable non-aqueous liquid formulation, such as those described above, can be employed along with any aqueous liquid formulation, such as those described above, provided that the mixed aqueous/non-aqueous liquid formulation provides stability to the compound contained therein. Preferably, the non-aqueous liquid in such a formulation is a hydrophilic liquid. Illustrative examples of suitable non-aqueous liquids include: glycerol; DMSO; EMS; ethylene glycols, such as PEG 200, PEG 300, and PEG 400; and propylene glycols, such as PPG 425, PPG 725, PEG 1000, PEG 2000, PEG 3000 and PEG 4000. Suitable stable formulations can permit storage of the active agents in a frozen or an unfrozen liquid state. Stable liquid formulations can be stored at a temperature of at least −70° C., but can also be stored at higher temperatures of at least 0° C., or between about 0° C. and about 42° C., depending on the properties of the composition. It is generally known to the skilled artisan that proteins and polypeptides are sensitive to changes in pH, temperature, and a multiplicity of other factors that may affect therapeutic efficacy.

In certain embodiments a desirable route of administration can be by pulmonary aerosol. Techniques for preparing aerosol delivery systems containing polypeptides are well known to those of skill in the art. Generally, such systems should utilize components that will not significantly impair the biological properties of the antibodies, such as the paratope binding capacity (see, for example, Sciarra and Cutie, “Aerosols,” in Remington's Pharmaceutical Sciences 18th edition, 1990, pp 1694-1712; incorporated by reference). Those of skill in the art can readily modify the various parameters and conditions for producing polypeptide aerosols without resorting to undue experimentation.

Other delivery systems can include time-release, delayed release or sustained release delivery systems. Such systems can avoid repeated administrations of compositions of the invention, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates polycaprolactones, polyesteramides, polyorthoesters, poiyhydroxybutyric acids, such as poly-D-(-)-3-hydroxybutyric acid (European Patent No. 133,988), copolymers of L-glutamic acid and gamma-ethyl-L-glutamate (Sidman, K R. et al, Biopolymers 22: 547-556), poly (2-hydroxyethyl methacrylate) or ethylene vinyl acetate (Langer, et al., J. Biomed. Mater. Res. 15:267-277; Langer, B. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono-, di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially filled implants; and the like. Specific examples include, but are not limited to: (a) aerosional systems in which the agent is contained in a form within a matrix such as those described in 13.5. U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480.

Another type of delivery system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μm, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Natl. Acad. Sci. (USA) 82:3688-3692 (1985); K. Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241.

Another type of vehicle is a biocompatible microparticle or implant that is suitable for implantation into the mammalian recipient. Exemplary bioerodible implants that are useful in accordance with this method are described in PCT International application no. PCT/US/03307 (Publication No-WO 95/24929, entitled “Polymeric Gene Delivery System”). PCT/US/0307 describes biocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle such as a microsphere (wherein an agent is dispersed throughout a solid polymeric matrix) or a microcapsule (wherein an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and composition are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time. The delivery system can also be a biocompatible microsphere that is suitable for local, site-specific delivery. Such microspheres are disclosed in Chickering, D. B., et al., Biotechnot. Bioeng, 52: 96-101; Mathiowitz, B., et al., Nature 386: 410-414.

Both non-biodegradable and biodegradable polymeric matrices can be used to deliver the compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluoses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate), poly(vinyl chloride), polystyrene, poly(viny lpyrrolidone), and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Methods of Ocular Delivery

The compositions of the invention are particularly suitable for treating ocular diseases or conditions, such as light toxicity, in particular light toxicity related to an ocular surgical procedure.

In one approach, the compositions of the invention are administered through an ocular device suitable for direct implantation into the vitreous of the eye. The compositions of the invention may be provided in sustained release compositions, such as those described in, for example, U.S. Pat. Nos. 5,672,659 and 5,595,760. Such devices are found to provide sustained controlled release of various compositions to treat the eye without risk of detrimental local and systemic side effects. An object of the present ocular method of delivery is to maximize the amount of drug contained in an intraocular device or implant while minimizing its size in order to prolong the duration of the implant. See, e.g., U.S. Pat. Nos. 5,378,475; 6,375,972, and 6,756,058 and U.S. Publications 20050096290 and 200501269448. Such implants may be biodegradable and/or biocompatible implants, or may be non-biodegradable implants.

Biodegradable ocular implants are described, for example, in U.S. Patent Publication No. 20050048099. The implants may be permeable or impermeable to the active agent, and may be inserted into a chamber of the eye, such as the anterior or posterior chambers or may be implanted in the sclera, transchoroidal space, or an avascularized region exterior to the vitreous. Alternatively, a contact lens that acts as a depot for compositions of the invention may also be used for drug delivery.

In a preferred embodiment, the implant may be positioned over an avascular region, such as on the sclera, so as to allow for transcleral diffusion of the drug to the desired site of treatment, e.g. the intraocular space and macula of the eye. Furthermore, the site of transcleral diffusion is preferably in proximity to the macula. Examples of implants for delivery of a composition of the invention include, but are not limited to, the devices described in U.S. Pat. Nos. 3,416,530; 3,828,777; 4,014,335; 4,300,557; 4,327,725; 4,853,224; 4,946,450; 4,997,652; 5,147,647; 164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146.366; 6,251,090; and 6,299,895, and in WO 01/30323 and WO 01/28474, all of which are incorporated herein by reference.

Examples include, but are not limited to the following: a sustained release drug delivery system comprising an inner reservoir comprising an effective amount of an agent effective in obtaining a desired local or systemic physiological or pharmacological effect, an inner tube impermeable to the passage of the agent, the inner tube having first and second ends and covering at least a portion of the inner reservoir, the inner tube sized and formed of a material so that the inner tube is capable of supporting its own weight, an impermeable member positioned at the inner tube first end, the impermeable member preventing passage of the agent out of the reservoir through the inner tube first end, and a permeable member positioned at the inner tube second end, the permeable member allowing diffusion of the agent out of the reservoir through the inner tube second end; a method for administering a compound of the invention to a segment of an eye, the method comprising the step of implanting a sustained release device to deliver the compound of the invention to the vitreous of the eye or an implantable, sustained release device for administering a compound of the invention to a segment of an eye; a sustained release drug delivery device comprising: a) a drug core comprising a therapeutically effective amount of at least one first agent effective in obtaining a diagnostic effect or effective in obtaining a desired local or systemic physiological or pharmacological effect; b) at least one unitary cup essentially impermeable to the passage of the agent that surrounds and defines an internal compartment to accept the drug core, the unitary cup comprising an open top end with at least one recessed groove around at least some portion of the open top end of the unitary cup; c) a permeable plug which is permeable to the passage of the agent, the permeable plug is positioned at the open top end of the unitary cup wherein the groove interacts with the permeable plug holding it in position and closing the open top end, the permeable plug allowing passage of the agent out of the drug core, though the permeable plug, and out the open top end of the unitary cup; and d) at least one second agent effective in obtaining a diagnostic effect or effective in obtaining a desired local or systemic physiological or pharmacological effect; or a sustained release drug delivery device comprising: an inner core comprising an effective amount of an agent having a desired solubility and a polymer coating layer, the polymer layer being permeable to the agent, wherein the polymer coating layer completely covers the inner core.

Other approaches for ocular delivery include the use of liposomes to target a compound of the present invention to the eye, and preferably to retinal pigment epithelial cells and/or Bruch's membrane. For example, the compound maybe complexed with liposomes in the manner described above, and this compound/liposome complex injected into patients with an ophthalmic condition, such as light toxicity, using intravenous injection to direct the compound to the desired ocular tissue or cell. Directly injecting the liposome complex into the proximity of the retinal pigment epithelial cells or Bruch's membrane can also provide for targeting of the complex with some forms of ocular PCD. In a specific embodiment, the compound is administered via intra-ocular sustained delivery (such as VITRASERT or ENVISION. In a specific embodiment, the compound is delivered by posterior subtenons injection. In another specific embodiment, microemulsion particles containing the compositions of the invention are delivered to ocular tissue to take up lipid from Bruchs membrane, retinal pigment epithelial cells, or both.

Nanoparticles are a colloidal carrier system that has been shown to improve the efficacy of the encapsulated drug by prolonging the serum half-life. Polyalkylcyanoacrylates (PACAs) nanoparticles are a polymer colloidal drug delivery system that is in clinical development, as described by Stella et al, J. Pharm. Sci., 2000. 89: p. 1452-1464; Brigger et al., Tnt. J. Pharm., 2001. 214: p. 37-42; Calvo et al., Pharm. Res., 2001. 18: p. 1157-1166; and Li et al., Biol. Pharm. Bull., 2001.24: p. 662-665. Biodegradable poly (hydroxyl acids), such as the copolymers of poly (lactic acid) (PLA) and poly (lactic-co-glycolide) (PLGA) are being extensively used in biomedical applications and have received FDA approval for certain clinical applications. In addition, PEG-PLGA nanoparticles have many desirable carrier features including (i) that the agent to be encapsulated comprises a reasonably high weight fraction (loading) of the total carrier system; (ii) that the amount of agent used in the first step of the encapsulation process is incorporated into the final carrier (entrapment efficiency) at a reasonably high level; (iii) that the carrier have the ability to be freeze-dried and reconstituted in solution without aggregation; (iv) that the carrier be biodegradable; (v) that the carrier system be of small size; and (vi) that the carrier enhance the particles persistence.

Nanoparticles are synthesized using virtually any biodegradable shell known in the art. In one embodiment, a polymer, such as poly (lactic-acid) (PLA) or poly (lactic-co-glycolic acid) (PLGA) is used. Such polymers are biocompatible and biodegradable, and are subject to modifications that desirably increase the photochemical efficacy and circulation lifetime of the nanoparticle. In one embodiment, the polymer is modified with a terminal carboxylic acid group (COOH) that increases the negative charge of the particle and thus limits the interaction with negatively charge nucleic acid aptamers. Nanoparticles are also modified with polyethylene glycol (PEG), which also increases the half-life and stability of the particles in circulation. Alternatively, the COOH group is converted to an N-hydroxysuccinimide (NHS) ester for covalent conjugation to amine-modified aptamers.

Biocompatible polymers useful in the composition and methods of the invention include, but are not limited to, polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, poly(viny Ipyrrolidone), polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt poly-methyl methacrylate), poly(ethyl methacrylate), poly(butyl methacrylate), poly(isobutyl methacrylate\ poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), polyvinyl acetate, polyvinyl chloride polystyrene, poly(vinyl pyrrolidone), polyhyaluronic acids, casein, gelatin, glutin, polyanhydrides, polyacrylic acid, alginate, chitosan, poly(methyl methacrylates), poly(ethyl methacrylates), poly(butyl methacrylate), poly(isobutyl methacrylate), poly(hexyl methacrylate) poly(isodecyl methaerylate), poly(lauryl methacrylate), poly(phenyl methacrylate), poly(methyl acrylate), poly(isopropyl acrylatee), poly(isobutyl acrylate), poly(octadecyl acrylate) and combinations of any of these, In one embodiment, the nanoparticles of the invention include PEG-PLGA polymers.

Compositions of the invention may also be delivered topically. For topical delivery, the compositions are provided in any pharmaceutically acceptable excipient that is approved for ocular delivery. Preferably, the composition is delivered in drop form to the surface of the eye. For some application, the delivery of the composition relies on the diffusion of the compounds through the cornea to the interior of the eye.

Those of skill in the art will recognize that treatment regimens for using the compounds of the present invention to treat light toxicity or other opthalmic conditions (e.g., retinitis pigmentosa) can be straightforwardly determined. This is not a question of experimentation, but rather one of optimization, which is routinely conducted in the medical arts. In vivo studies in nude mice often provide a starting point from which to begin to optimize the dosage and delivery regimes. The frequency of injection will initially be once a week, as has been done in some mice studies. However, this frequency might be optimally adjusted from one day to every two weeks to monthly, depending upon the results obtained front the initial clinical trials and the needs of a particular patient.

Human dosage amounts can initially be determined by extrapolating from the amount of compound used in mice, as a skilled artisan recognizes it is routine in the art to modify the dosage for humans compared to animal models. For certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose maybe about 1, 5, 10, 25, 50, 75, 100, 150, 10 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that lower does may be used, such doses may be in the range of about 5 mg compound/Kg body to about 20 mg compound/Kg body. In other embodiments the doses may be about 8, 10, 12, 14, 16 15 or 18 mg/Kg body weight. Of course, this dosage amount may be adjusted upward or downward, as is routinely done in such treatment protocols, depending on the results of the initial clinical trials and the needs of a particular patient.

Screening Assays

Useful compounds of the invention are non-retinoids that reversibly bind non-covalently to a native opsin protein, such as in or near the retinal binding pocket, to prevent light toxicity related to, for example, the accumulation of visual cycle products. Such binding will commonly inhibit, if not prevent, binding of retinoids, especially 11-cis-retinal, to the binding pocket and thereby reduce formation of visual cycle products, such as all-trans-retinal. Any number of methods are available for carrying out screening assays to identify such compounds. In one approach, an opsin protein is contacted with a candidate compound or test compound that is a non-retinoid in the presence of 11-cis-retinal or retinoid analog and the rate or yield offormation of chromophore is determined. If desired, the binding of the non-retinoid to opsin is characterized. Preferably, the non-retinoid binding to opsin is non-covalent and reversible b. Thus, inhibition of rhodopsin formation by a non-retinoid indicates identification of a successful test compound. An increase in the amount of rhodopsin is assayed, for example, by measuring the protein's absorption at a characteristic wavelength (e.g., 498 nm for rhodopsin) or by measuring an increase in the biological activity of the protein using any standard method (e.g., enzymatic activity association with a ligand). Useful compounds inhibit binding of 11-cis-retinal (and formation of rhodopsin) by at least about 10%, 15%, or 20%, or preferably by 25%, 50%, or 75%, or most preferably by up to 90% or even 100%.

The efficacy of the identified compound is assayed in an animal model showing the effects of light toxicity. For example, the efficacy of compounds disclosed herein have been demonstrated using transgenic mice that contain a mutant elaov 4 gene important in fatty acid synthesis and transgenic mice that produce a mutant ABCR protein that affects how all-trans-retinal is shuttled. The amount of lipofuscin produced in such mice was determined using compounds of the invention and shown to be produced at a reduced rate resulting in slower accumulation of toxic visual cycle products. In either case, the cellular phenotype is the same and lipofuscin is accumulated at an accelerated rate when successful test compounds are not administered.

Alternatively, the efficacy of compounds useful in the methods of the invention may be determined by exposure of a mammalian eye to a high intensity light source prior to, during, or following administration of a test compound, followed by determination of the amount of visual cycle products (e.g., all-trans retinal, A2E, or lipofuscin) formed as a result of exposure to the high intensity light source, wherein a compound of the invention will have reduced the amount of visual cycle products related to the exposure.

In sum, preferred test compounds identified by the screening methods of the invention are non-retinoids, are selective for opsin and bind in a reversible, non-covalent manner to opsin protein. In addition, their administration to transgenic animals otherwise producing increased lipofuscin results in a reduced rate of production or a reduced accumulation of lipofuscin in the eye of said animal. Compounds identified according to the methods of the invention are useful for the treatment of light toxicity or other ophthalmic condition in a subject, such as a human patient.

Test Compounds and Extracts

In general, compounds capable of decreasing the formation of visual cycle products, such as all-trans-retinal, either in vitro or in vivo, are identified from large libraries of either natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangaphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonoimc dereplication, biological dereplication, and chemical dereplication, or any combination thereof) or the elimination of replicates or repeats of materials already known for their activity in reducing formation of visual cycle products should be employed whenever possible.

When a crude extract is found to reduce formation of visual cycle products or to compete reversibly with 11-cis-retinal for binding at, near or in the retinal binding pocket of opsin protein, further fractionation of the positive lead extract is necessary to isolate chemical constituents responsible for the observed effect. Thus, the goal of the extraction, fractionation, and purification process is the careful characterization and identification of a chemical entity within the crude extract that increase the yield of a correctly folded protein. Methods of fractionation and purification of such heterogeneous extracts are known in the art. If desired, non-retinoid compounds shown to be useful agents for the treatment of any pathology related to the visual cycle are chemically modified according to methods known in the art.

Combination Therapies

Compositions of the invention useful for the prevention of light toxicity can optionally be combined with additional therapies as heretofore described.

### EXAMPLES

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The invention is described in more detail in the following non-limiting examples. It is to be understood that these methods and examples in no way limit the invention to the embodiments described herein and that other embodiments and uses will no doubt suggest themselves to those skilled in the art.

### Reagents

Small molecules were procured from National Cancer Institute. Monoclonal anti-rhodopsin 1D4 antibody was purchased from University of British Columbia. j3-Ionone was from Sigma and Dodecylmaltopyrannoside (DM) was procured from Anatrace.

Database preparation.

The National Cancer Institute/Developmental Therapeutics Program (NCI/DTP) maintains a repository of approximately 220,000 samples (the plated compound set) which are non-proprietary and offered to the extramural research community for the discovery and development of new agents for the treatment of cancer, AIDS, or opportunistic infections afflicting patients with cancer or AIDS (Monga and Sausville 2002). The three dimensional coordinates for the NCI/DTP plated compound set was obtained in the MDL SD format and converted to the mol2 format by the DOCK utility program SDF2MOL2 (UCSF). Partial atomic charges, solvation energies and van der Waals parameters for the ligands were calculated using SYBDB (Tripos, Inc.) and added to the plated compound set mol2 file.

### Molecular Docking

All docking calculations were performed with the Oct. 15, 2002, development version of DOCK, v5.1.0 (Charifson et al. 1999; Ewing et al. 2001). The general features of DOCK include rigid orienting of ligands to receptor spheres, AMBER energy scoring, GB/SA solvation scoring, contact scoring, internal non-bonded energy scoring, ligand flexibility and both rigid and torsional simplex minimization (Gschwend et al.; Good et al. 1995). Unlike previously distributed versions, this release incorporates automated matching, internal energy (used in flexible docking), scoring function hierarchy and new minimizer termination criteria.

The coordinates for the crystal structure of rhopdopsin, PDB code 1 GZM, was used in the molecular docking calculations. To prepare the site for docking, all water molecules were removed. Protonation of receptor residues was performed with Sybyl (Tripos, St. Louis, Mo.). The structure was explored using sets of spheres to describe potential binding pockets. The number of orientations per molecule was 100. Intermolecular AMBER energy scoring (vdw+columbic), contact scoring and bump filtering were implemented in DOCK5.1.0 (Gschwend et al,). SETOR (Evans 1993) and GRASP (Petrey and Honig 2003) were used to generate molecular graphic images.

### Cell Lines and Culture Conditions

Stable cell lines expressing opsin protein were generated using the Flp-In T-Rex system. The stable cells were grown in DMEM high glucose media supplemented with 10% (vlv) fetal bovine serum, antibiotic/antimycotic solution, 5 μL/ml blasticidin and hygromycin at 37° C. in presence of 5% CO₂. For all the experiments the cells were allowed to reach confluence and were induced to produce opsin with 1 μg/ml tetracycline after change of media and then compounds were added. The plates were incubated for 48 hours after which the cells were harvested.

### SDS-PAGE and Western Blotting

Proteins were separated on SDS-PAGE gels and western blotted as described in Noorwez et al. (2004).

### Example 1

### Use of a Crystal Structure of Rhodopsin To Select Potential Modulators

The retinal binding pocket of a trigonal crystal form of bovine rhodopsin, PDB code 1 GZM, was used to identify small molecule modulators by a high throughput molecular docking method. The positions of each retinal atom were used to guide in the definition of the binding pocket selected for molecular docking.

Spheres were positioned at the selected site to allow the molecular docking program, DOCK 5. 1.0 (available from USCF), to match spheres with atoms in potential ligands (small molecules in this case). During the molecular docking calculation, orientations are sampled to match the largest number of spheres to potential ligand atoms, looking for the low energy structures that bind tightly to the active site of a receptor or enzyme whose active site structure is known.

A scoring grid was calculated to estimate the interaction between potential ligands and the retinal binding pocket target site. The atomic positions and chemical characteristics of residues in close proximity (within 4 angstroms (Å)) to the selected site were used to establish a scoring grid to evaluate potential interactions with small molecules. Two types of interactions were scored: van der Waals contact and electrostatic interactions.

DOCKS.1.0 was used to carry out docking molecular dynamic simulations. The coordinates for approximately 20,000 drug-like compounds (all of which are available through the National Cancer Institute/DTP) were used as the ligand database for molecular docking using the site selected (the retinal binding pocket). These 20,000 compounds were selected from the NCI/DTP collection based on the Lipinski rules for drug likeness. Each small molecule was positioned in the selected site in 100 different orientations, and the best orientations and their scores (contact and electrostatic) were calculated. The scored compounds were ranked and the 20 highest scoring compounds were requested from the NCI/DTP for functional evaluation.

D. Research Design and Methods

D.1 Database Preparation

The National Cancer Institute/Developmental Therapeutics Program (NCI/DTP) maintains a repository of approximately 220,000 samples (the plated compound set) which are non-proprietary and offered to the extramural research community for the discovery and development of new agents for the treatment of cancer, AIDS, or opportunistic infections afflicting patients with cancer or AIDS (Monga and Sausville (2002)). The three-dimensional coordinates for the NCI/DTP plated compound set was obtained in the MDL SD format and converted to the mol2 format by the DOCK utility program SDF2MOL2 ((UCSF). Partial atomic charges, solvation energies and van der Waals parameters for the ligands were calculated using SYBDB (Tripos, Inc.) and added to the plated compound set mol2 file).

D.2 Molecular Docking

All docking calculations were performed with the Oct. 15, 2002, development version of DOCK, v5.1.0 (Charifson et al. 1999; Ewing et al. 2001). The general features of DOCK include rigid orienting of ligands to receptor spheres, AMBER energy scoring, GB/SA salvation scoring, contact scoring, internal non-bonded energy scoring, ligand flexibility and both rigid and torsional simplex minimization (Gschwend et al.; Good et al. 1995). Unlike previously distributed versions, this release incorporates automated matching, internal energy (used in flexible docking), scoring function hierarchy and new minimizer termination criteria.

The coordinates for the crystal structure of rhodopsin, PDB code 1 GZM, were used in the molecular docking calculations. To prepare the site for docking, all water molecules were removed. Protonation of receptor residues was performed with Sybyl (Tripos, St. Louis, Mo.). The structure was explored using sets of spheres to describe potential binding pockets. The number of orientations per molecule was 100. Intermolecular AMBER energy scoring (vdw+columbic), contact scoring and bump filtering were implemented in DOCK 5.1.0 (Gschwend el.). SETOR (Evans 1993) and GRASP (Petrey and Honig 2003) were used to generate molecular graphic images.

Compounds showing activity in reversible binding to opsin and inhibition of 11-cis-retinal binding include such structure as:

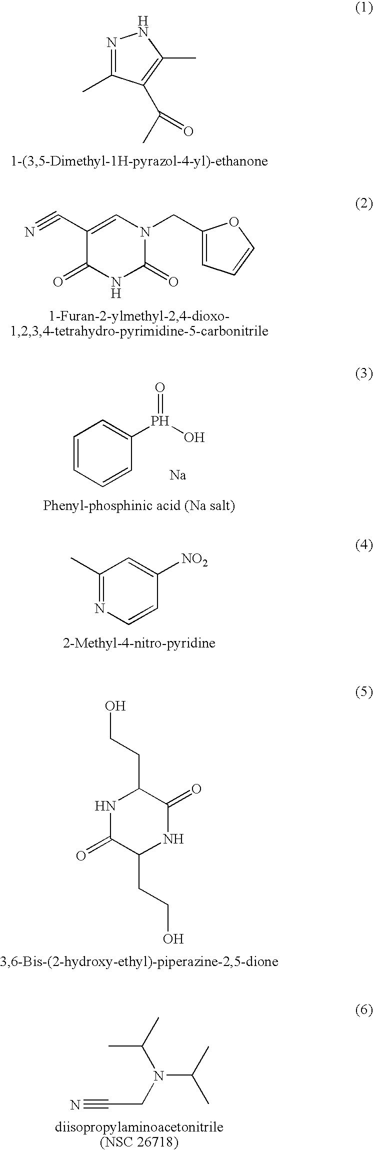

### Example 2

### Effect of β-Ionone on Opsin-Binding of 11-cis-Retinal

The structure of β-ionone is as follows:

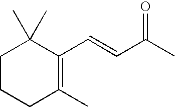

As shown in FIG. 1, to determine whether a 500 nm absorbing pigment is formed upon addition of β-ionone, purified wt (wild-type) opsin was mixed with β-ionone, incubated for 15 minutes, and scanned for pigment formation. β-ionone does not form a light absorbing pigment with opsin.

Here we have demonstrated that smaller molecules, e.g., β-ionone, that non-covalently bind to the chromophore binding site of opsin, inhibit binding of retinal to the site and thereby reduce formation of visual cycle products, such as all-trans-retinal. Similar results have been found for cis-1,3-dimethylcyclohexane. It is important to note that these compounds are non-retinoids. We have utilized a high-throughput computer-based molecular docking approach that made use of the coordinates of the retinal binding site coupled with functional studies in vitro and in vivo to identify 1-(3,5-dimethyl-1H-pyrazol-4-yl)ethanone (SN 10011), a drug-like small molecule, that inhibits the binding of 11-cis-retinal to opsin in vitro, suggesting that the identified molecules occupy the retinal binding pocket. Although the molecular docking strategy is a powerful tool for the discovery of selective inhibitors, the present invention demonstrates a novel utility for the power of high-throughput in silico screening combined with functional testing in identifying novel pharmacological chaperones. Such functional testing is recited in the screening methods of the invention. Thus, in silico methods have proved useful in identifying types of non-retinoid molecules that might prove useful in inhibiting binding of retinoids and reducing formation of visual cycle products such as all-trans-retinal. Once identified, these compounds exhibited selective binding properties in their interaction with opsin and the screening methods of the invention take advantage of these properties to find other compounds binding through a similar mechanism as a means of identifying potential therapeutic agents.

Collectively, these results indicate that small compounds that fit into the retinal binding pocket of opsin and compete with 11-cis-retinal in vitro are good therapeutic agents for the methods of the invention.

### Example 3

### Effect of SN10011 on Opsin Regeneration

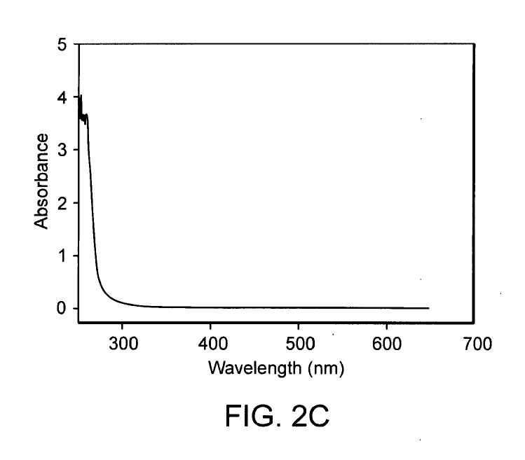

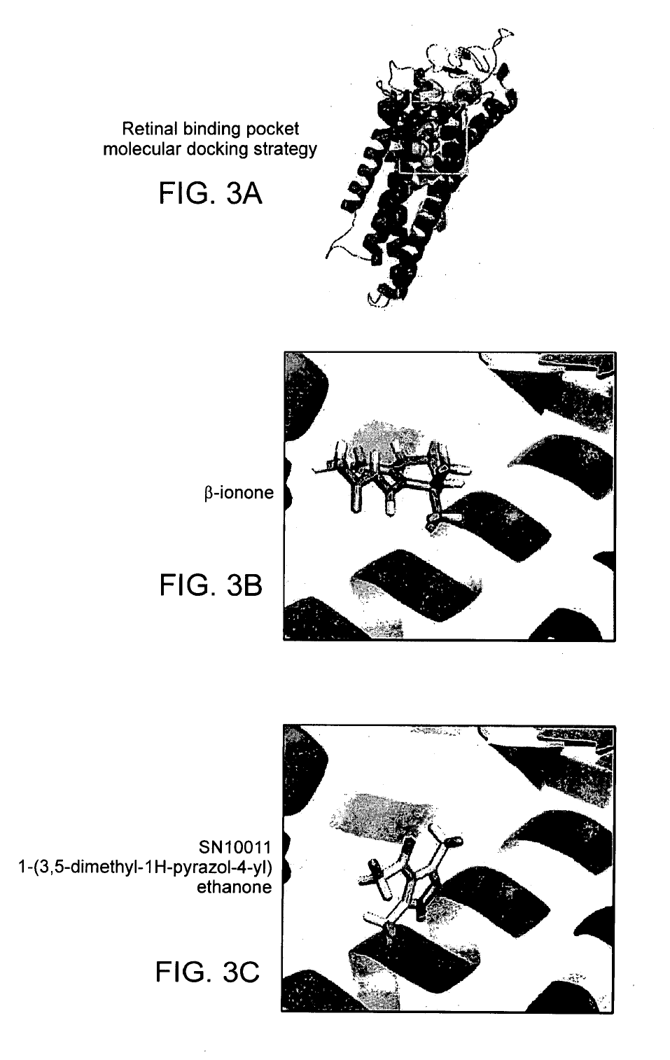

To identify non-retinoid compounds that are useful therapeutic agents, we performed molecular docking using a large chemical library of drug-like small molecules in the National Cancer Institute Developmental Therapeutics Program. DOCK 5.1.0 (UCSF) was used to position each one of 20,000 drug-like compounds into the selected site. Each compound was positioned in 100 different orientations, and the best scoring orientations were obtained, Unlike previous molecular docking strategies, each docked compound was selected based on chemical criteria (for example, the Lipinski rules for drug likeness). Therefore, this strategy eliminates compounds that are less likely to be developed into therapeutic agents. FIG. 3C shows results with the 5th highest scoring compound, 1-(3,5-dimethyl-1-H-pyrazol-4-yl)ethanone, SN10011, in the orientation posed by DOCK 5.1.0 (UCSF) at the retinal binding pocket based on the crystal structure of rhodopsin. Compound SN10011 reversibly inhibits binding of 11-cis-retinal.

We tested the top scoring compounds (the highest 0.05% energy scores) for their effect as inhibitors of retinoid binding. One compound, SN10011 showed a significant effect on inhibition of pigment formation with 11-cis-retinal. The effect of SN10011 was studied by addition of 2 and 5 mM SN10011 to the opsin solution followed by addition of 11-cis-retinal (FIG. 2a). Presence of this compound increased the t_{1/2} from 5 minutes to 8 minutes (2 mM) and 12 minutes (5 mM), respectively. This demonstrates a dose dependence of regeneration inhibition. The extent of inhibition was much lower than that obtained with β-ionone and the concentrations of this compounded needed to reach the observable inhibition levels were also much higher than that of β-ionone. To test whether this compound associates with wild-type (WT) opsin to form pigment, it was added to opsin solution in vitro. No pigment was formed by SN10011 with WT opsin (FIG. 2b) and by itself the compound did not show any absorption in the visible spectrum (FIG. 2c).

Thus, we have utilized a high-throughput computer-based molecular docking approach that made use of the coordinates of the retinal binding site coupled with functional studies in vitro and in vivo to identify 1-(3,5-dimethyl-1H-pyrazol-4-yl)ethanone (SN 10011), a drug-like small molecule, that inhibits the binding of 11-cis-retinal to opsin in vitro, suggesting that the identified molecules occupy the retinal binding pocket.

These results suggest that the contacting of such compounds with opsin in the eye of a mammal serves to inhibit binding of 11-cis-retinal in the binding pocket of the protein and thereby reduce formation of products like all-trans-retinal, leading to reduced formation of toxic materials like lipofuscin and retarding, if not completely preventing, the progression toward maladies such as light toxicity.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof. All patents and publications mentioned in this specification are herein incorporated by reference to the same extent as if each independent patent and publication was specifically and individually indicated to be incorporated by reference.

### REFERENCES

Abagyan et al., Curr. Opin. in Chemical Biology 5(4): 375-82 (2001).
Bernier et al., Trends Endocrinol Metab 15, 222-228 (2004).
Berson, E. L. Int. Opthalmol Clin. 40, 93-111 (2000).
Boehm et al., J. Med. Chem. 43(14): 2664-74 (2000).
Charifson et al., Journal of Medicinal Chemistry 42(25): 5100-9 (1999).
Collins et al., Annu. Rev. Pharmacol. Toxicol. 39, 399-430 (1999).
Doman et al., J. Med. Chem. 45(11): 2213-21 (2002).
Enyedy et al., J. Med. Chem. 44(9): 1349-55 (2001).
Enyedy et al., J. Med. Chem. 44(25): 4313-24 (2001).
Evans, S. V., J. Mol. Graphics. 11(2): 134-8, 127-8 (1993).
Ewing et al., Journal of Computer-Aided Molecular Design 15(5): 411-28 (2001).
Freymann et al., Chemistry & Biology 7(12): 957-68 (2000).
Good et al., Journal of Computer-Aided Molecular Design 9(1): 1-12 (1995).
Gruneberg et al., J. Med. Chem. 45(17): 3588-602 (2002).
Gschwend et al., Journal of Molecular Recognition 9(2): 175-86 (1996).
Honma et al., J. Med. Chem. 44(26): 4615-27 (2001).
Honma et al., J. Med. Chem. 44(26): 4628-40 (2001).
Iwata et al., J. Med. Chem. 44(11): 1718-28 (2001).
Krebs et al., Trends Biochem. Sci. 29, 648-655 (2004).
Kuksa et al., J. Biol. Chem. 277, 42315-42324 (2002).
Li et al., Proc. Natl. Acad. Sci. (USA) 95, 11933-11938 (1998).
Liebeschuetz et al., J. Med. Chem. 45(6): 1221-32 (2001).
Monga et al., Leukemia: Official Journal of the Leukemia Society of America, Leukemia Research Fund, U.K 16(4): 520-6 (2002).
Noorwez et al., J. Biol. Chem. 278, 14442-14450 (2003).
Noorwez et al., J. Biol. Chem. 279, 16278-16284 (2004).
Paiva et al., Biochimica Biophysica Acta 1545(1-2): 67-77 (2001).
Pang et al., FEBS Letters 502(3): 93-97 (2001).
Petrey et al., Meth. Enzym. 374:492-509 (2003)
Saliba et al., J. Cell Sci., 115, 2907-2918 (2002).
Schapira et al., PNAS 97(3): 1008-13 (2000).
Shoichet et al., Curr. Opin. in Chem. Biol. 6(4): 439-46 (2002).
Teelmann et al., Pharmacol. Ther. 40, 29-43 (1989).

1. A method of reducing light toxicity in a mammalian eye, comprising administering to said mammal an opsin-binding agent that
(a) is a retinoid that binds non-covalently to said opsin protein; or
(b) is a non-retinoid that binds reversibly to said opsin protein; thereby reducing light toxicity in said mammalian eye.

2. The method of claim 1, wherein said retinoid or non-retinoid opsin-binding agent selectively binds to opsin.

3. The method of claim 1, wherein said opsin-binding agent is a non-retinoid.

4. The method of claim 1, wherein said opsin binding agent binds at or near the retinal binding pocket of said opsin protein.

5. The method of claim 1, wherein said opsin-binding agent binds to said opsin protein so as to inhibit covalent binding of 11-cis-retinal to said opsin protein when said 11-cis-retinal is contacted with said opsin protein in the presence of said non-retinoid opsin-binding agent.

6. The method of claim 1, wherein said opsin-binding agent binds to said opsin in the retinal binding pocket of opsin protein or disrupts retinoid binding to the retinal binding pocket of opsin.

7. The method of claim 1, wherein said opsin-binding agent binds to said opsin protein so as to inhibit covalent binding of 11-cis-retinal to said opsin protein.

8. The method of claim 1, wherein said mammal is a human being.

9. The method of claim 1, wherein said light toxicity is associated with the level of a visual cycle product.

10. The method of claim 6, wherein said visual cycle product is a product formed from 11-cis-retinal or all-trans-retinal.

11. The method of claim 6, wherein said visual cycle product is a toxic visual cycle product.

12. (canceled)

13. The method of claim 6, wherein said visual cycle product is lipofuscin or N-retinylidene-N-retinylethanolamine (A2E).

14. (canceled)

15. The method of claim 1, wherein said administering is topical administration or systemic administration to said eye.

16-18. (canceled)

19. The method of claim 1, wherein said light toxicity is related to an ophthalmic procedure.

20. The method of claim 19, wherein said ophthalmic procedure is ophthalmic surgery.

21. The method of claim 20, wherein said administering occurs prior to, during, or after said ophthalmic surgery.

22-23. (canceled)

24. The method of claim 1, further comprising administering to said mammal an effective amount of at least one additional agent selected from the group consisting of a proteasomal inhibitor, an autophagy inhibitor, a lysosomal inhibitor, an inhibitor of protein transport from the ER to the Golgi, an Hsp90 chaperone inhibitor, a heat shock response activator, a glycosidase inhibitor, and a histone deacetylase inhibitor.

25-28. (canceled)

29. The method of claim 1, further comprising administering to said mammal at least one anti-inflammatory agent.

30-32. (canceled)

33. The method of claim 1, further comprising administering to said mammal at least one anti-oxidant selected from the group consisting of vitamin A, vitamin C and vitamin E.

34-35. (canceled)

36. The method of claim 1, wherein the opsin-binding agent is selected from the group consisting of 1-(3,5-dimethyl-1H-pyrazol-4-yl)-ethanone, 1-furan-2-ylmethyl-2,4-dioxo-1,2,3,4-tetrahydro-pyrimidine-5-carbonitrile, phenyl-phosphinic acid, 2-methyl-4-nitro-pyridine, 3,6-bis-(2-hydroxyethy)-piperazine-2,5-dione, diisopropylaminoacetonitrile, 3,4-methylenedioxybenzonitrile, diethyl(2-mercaptoethyl)amine, 6-imino-1-methyl-1,6-dihydro-3-pyridinecarboxamide, 1H-1,2,3-benzotriazol-1-amine, 4-salicylideneamino-1,2,4-triazole, β-ionone, cis-1,3-dimethylcyclohexane, and a pharmaceutically acceptable salt thereof.

37-45. (canceled)
